(19) United States
(12) Patent Application Publication
(10) Pub. No.: US 2026/0032825 A1
(43) Pub. Date: Jan. 29, 2026

(54) DETECTION DEVICE

(71) Applicant: Japan Display Inc., Tokyo (JP)

(72) Inventor: Atsunori OYAMA, Tokyo (JP)

(21) Appl. No.: 19/349,205

(22) Filed: Oct. 3, 2025

Related U.S. Application Data
(63) Continuation of application No. PCT/JP2024/013864, filed on Apr. 4, 2024.

(30) Foreign Application Priority Data
Apr. 12, 2023 (JP) 2023-065113

(51) Int. Cl. H05K 1/18

(52) U.S. Cl. CPC H05K 1/189; H05K 1/181; H05K 2201/10151

(57) ABSTRACT

A detection device includes: a substrate having opposite ends with a notch therebetween; a terminal part provided at one end of the substrate; a first optical sensor on the substrate between the notch and the terminal part; a second optical sensor on the substrate between the notch and another end of the substrate; and a flexible printed circuit board provided with a light source and electronic components. A lower electrode, a lower buffer layer, an active layer, an upper buffer layer, an upper electrode, and a sealing film are stacked on the substrate in each of the first and second optical sensors. The lower electrodes of the first and second optical sensors are coupled to the terminal part. The terminal part is coupled to an end of the flexible printed circuit board. The flexible printed circuit board is bent. The notch is disposed at a location overlapping the light source.

### CROSS-REFERENCE TO RELATED APPLICATION

This application claims the benefit of priority from Japanese Patent Application No. 2023-065113 filed on April 12, 2023 and International Patent Application No. PCT/JP2024/013864 filed on April 4, 2024, the entire contents of which are incorporated herein by reference.

### BACKGROUND

### 1. Technical Field

What is disclosed herein relates to a detection device.

### 2. Description of the Related Art

Devices that detect information on a living body from a human body are known. Japanese Patent Application Laid-open Publication No. 2012-065900 discloses a pulse wave sensor that can measure pulse waves without constraining actions of a subject person.

When a substrate including a plurality of optical sensors, a light source, and a battery are separately fixed and accommodated in an annular housing, the housing is made larger in size.

For the foregoing reasons, there is a need for a detection device that can be made smaller in size even when accommodating a substrate including optical sensors and members coupled to the substrate.

### SUMMARY

According to an aspect, a detection device includes: a substrate having opposite ends in a first direction with a notch interposed therebetween; a terminal part provided at one end in the first direction of the substrate; a first optical sensor provided on the substrate between the notch and the terminal part; a second optical sensor provided on the substrate between the notch and the other end of the substrate; and a flexible printed circuit board on which a light source and a plurality of electronic components are mounted. A lower electrode, a lower buffer layer, an active layer, an upper buffer layer, an upper electrode, and a sealing film are stacked on the substrate in the order as listed, in each of the first optical sensor and the second optical sensor. The lower electrodes of the first optical sensor and the second optical sensor are electrically coupled to the terminal part. The terminal part is coupled to a first end of the flexible printed circuit board. The flexible printed circuit board is bent, and the notch of the substrate is disposed at a location overlapping the light source.

According to an aspect, a detection device includes: a substrate provided with an optical sensor; a terminal part provided at one end in a first direction of the substrate; and a flexible printed circuit board on which a light source and a plurality of electronic components are mounted. A lower electrode, a lower buffer layer, an active layer, an upper buffer layer, an upper electrode, and a sealing film are stacked on the substrate in the order as listed, in the optical sensor. Each of the lower electrode and the upper electrode of the optical sensor is electrically coupled to the terminal part. The terminal part is coupled to a first end of the flexible printed circuit board. The flexible printed circuit board is bent, and the other end in the first direction of the substrate is located in a position adjacent to the light source.

### BRIEF DESCRIPTION OF THE DRAWINGS

FIG. 1 is a schematic view illustrating an exemplary external appearance when a state of a finger accommodated inside a detection device according to a first embodiment is viewed from a lateral side of a housing;

FIG. 2 is a schematic sectional view taken along section A-A illustrated in FIG. 1;

FIG. 3 is a development view illustrating an exemplary development of a front surface side of a substrate and a flexible printed circuit board illustrated in FIG. 2;

FIG. 4 is a development view illustrating an exemplary development of a back surface side of the substrate and the flexible printed circuit board illustrated in FIG. 2;

FIG. 5 is a development view illustrating a state where the flexible printed circuit board illustrated in FIG. 4 is bent;

FIG. 6 is a side view of the substrate and the flexible printed circuit board illustrated in FIG. 5;

FIG. 7 is a schematic sectional view illustrating an exemplary multilayer configuration of an optical sensor taken along section B-B illustrated in FIG. 3;

FIG. 8 is a schematic sectional view illustrating an exemplary multilayer configuration of the optical sensor taken along section C-C illustrated in FIG. 3;

FIG. 9 is a development view illustrating an exemplary development of the front surface side of a substrate and the flexible printed circuit board of a second embodiment;

FIG. 10 is a development view illustrating an exemplary development of the back surface side of the substrate and the flexible printed circuit board of the second embodiment;

FIG. 11 is a development view illustrating a state where the flexible printed circuit board illustrated in FIG. 10 is bent;

FIG. 12 is a side view of the substrate and the flexible printed circuit board illustrated in FIG. 11;

FIG. 13 is a development view illustrating an exemplary development of the front surface side of the substrate and the flexible printed circuit board of a third embodiment;

FIG. 14 is a development view illustrating an exemplary development of the back surface side of the substrate and the flexible printed circuit board of the third embodiment;

FIG. 15 is a development view illustrating a state where the flexible printed circuit board illustrated in FIG. 14 is bent; and

FIG. 16 is a side view of the substrate and the flexible printed circuit board illustrated in FIG. 15.

### DETAILED DESCRIPTION

The following describes modes (embodiments) for carrying out the disclosure in detail with reference to the drawings. The present disclosure is not limited to the description of the embodiments given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present specification and the drawings, and detailed description thereof may not be repeated where appropriate.

In the present specification and claims, in expressing an aspect of disposing another structure on or above a certain structure, a case of simply expressing "on" includes both a case of disposing the other structure immediately on the certain structure so as to contact the certain structure and a case of disposing the other structure above the certain structure with still another structure interposed therebetween, unless otherwise specified.

### First Embodiment

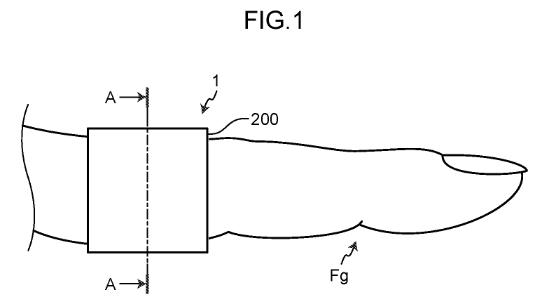

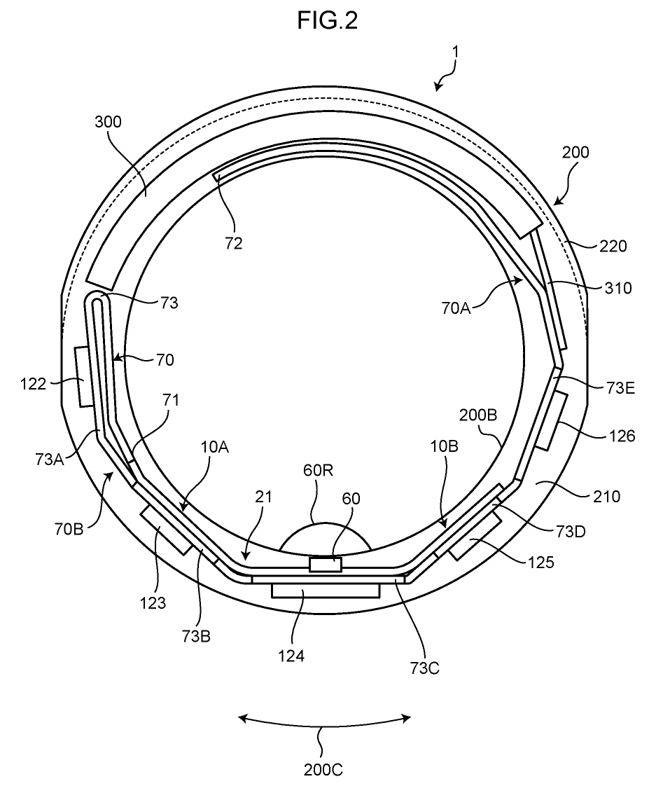

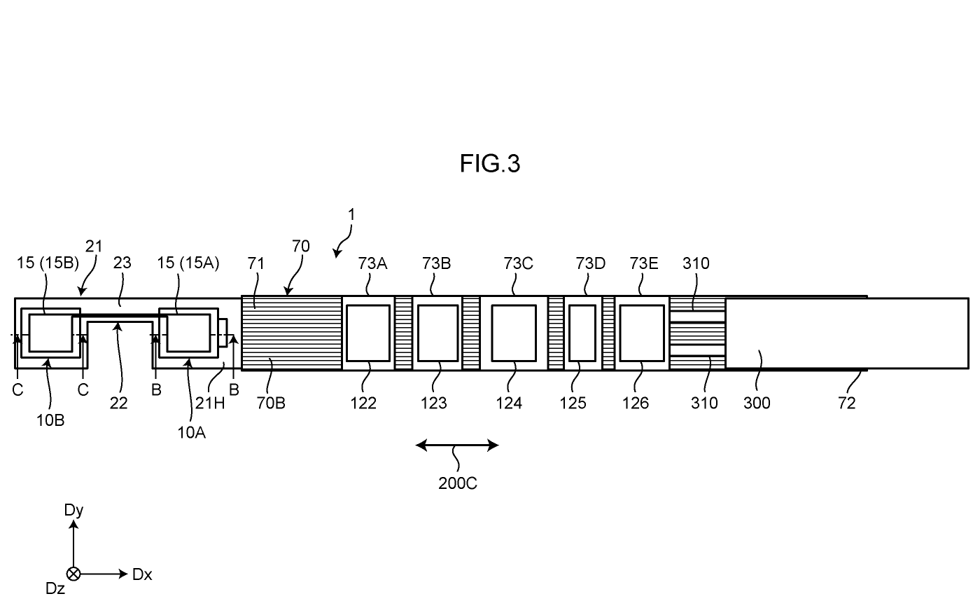

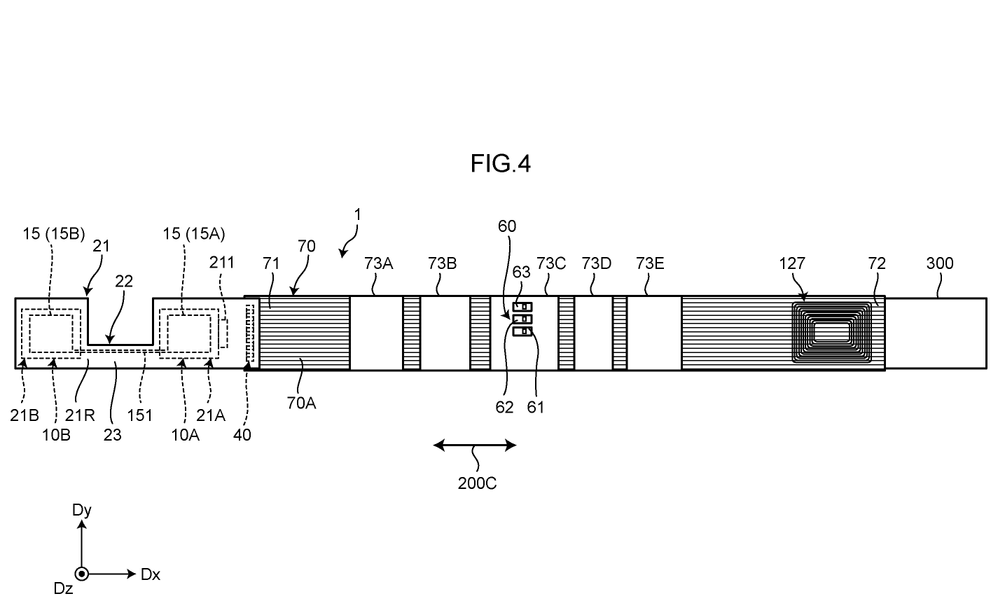

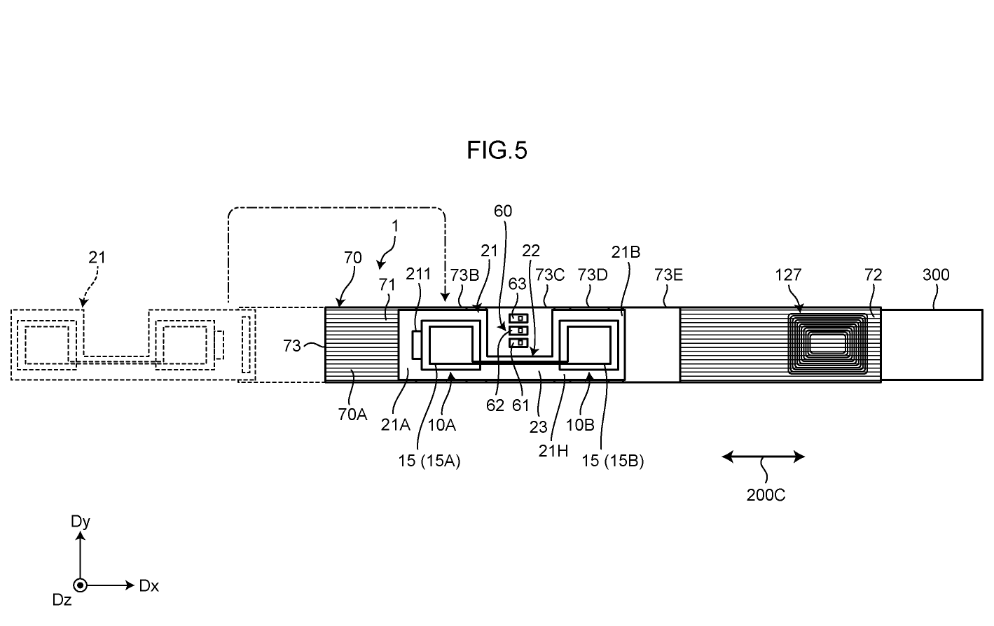

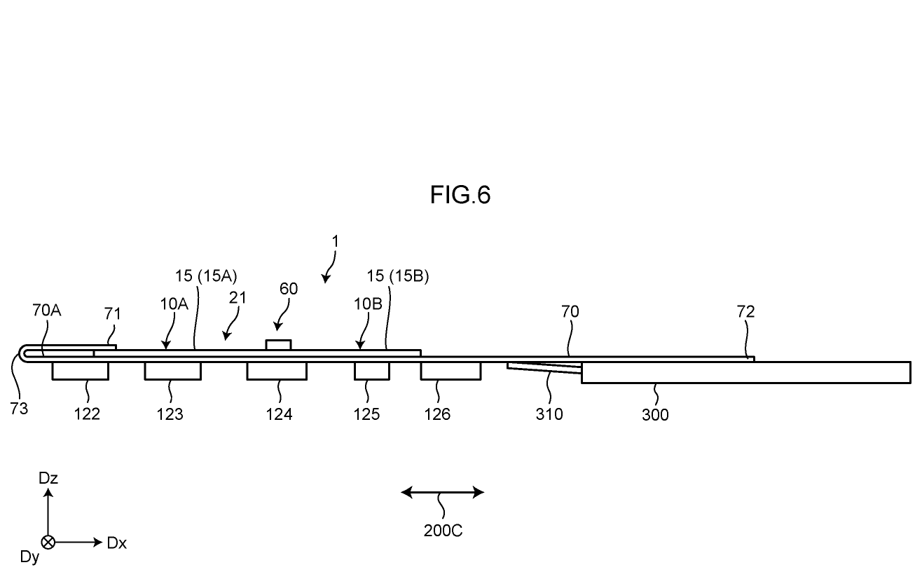

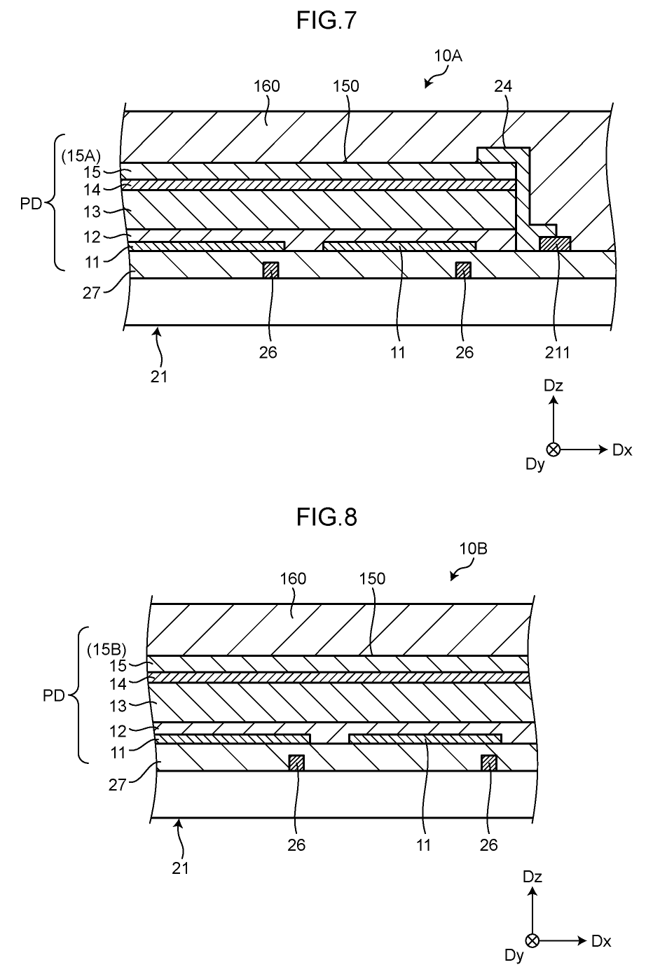

FIG. 1 is a schematic view illustrating an exemplary external appearance when a state of a finger accommodated inside a detection device according to a first embodiment is viewed from a lateral side of a housing. FIG. 2 is a schematic sectional view taken along section A-A illustrated in FIG. 1. FIG. 3 is a development view illustrating an exemplary development of a front surface side of a substrate and a flexible printed circuit board illustrated in FIG. 2. FIG. 4 is a development view illustrating an exemplary development of a back surface side of the substrate and the flexible printed circuit board illustrated in FIG. 2. FIG. 5 is a development view illustrating a state where the flexible printed circuit board illustrated in FIG. 4 is bent. FIG. 6 is a side view of the substrate and the flexible printed circuit board illustrated in FIG. 5. FIG. 7 is a schematic sectional view illustrating an exemplary multilayer configuration of an optical sensor taken along section B-B illustrated in FIG. 3. FIG. 8 is a schematic sectional view illustrating an exemplary multilayer configuration of the optical sensor taken along section C-C illustrated in FIG. 3.

A detection device 1 illustrated in FIG. 1 is a finger ring-shaped device that can be worn on and removed from a human body and is worn on a finger Fg of the human body. Examples of the finger Fg include a thumb, an index finger, a middle finger, a ring finger, and a little finger. The human body is a person to be authenticated whose identity is to be verified by the detection device 1. The detection device 1 can detect biometric information on a living body from the finger Fg wearing the detection device 1. The finger Fg is an example of a measurement target. The measurement target is the living body or a part of the living body, and is an object to be measured. The detection device 1 is made into a finger ring or a wristband so as to be easily carried by a user. In the following description, the detection device 1 is assumed to be used as a finger ring.

As illustrated in FIG. 2, the detection device 1 includes a housing 200, a substrate 21, a light source 60, a first optical sensor 10A, a second optical sensor 10B, and a flexible printed circuit board 70. The detection device 1 is a device that includes a battery 300 accommodated in the housing 200 and coupled to the flexible printed circuit board 70, and is operated by power of the battery 300. The substrate 21 is electrically coupled to the flexible printed circuit board 70.

The housing 200 is a mounting member that is formed in a ring shape (annular shape) wearable on the finger Fg, and is worn on the living body. In the example illustrated in FIG. 2, the housing 200 includes a first housing 210 and a second housing 220. The first housing 210 is integrated with the second housing 220 to form the housing 200 into the ring shape. The first housing 210 is a member that makes contact with the human body wearing the housing 200. The first housing 210 accommodates therein the light source 60, the first optical sensor 10A, the second optical sensor 10B, and so forth. The first housing 210 is formed into a ring shape using a housing material such as a light-transmitting synthetic resin or silicon. The first housing 210 includes a light emitter 60R that is convex inward, thus being configured to be capable of emitting light of the light source 60 from the light emitter 60R to the finger Fg. The light emitter 60R is a light-transmitting convex lens, for example.

The second housing 220 has a surface of the housing 200 that covers a part of an outer peripheral surface 210A of the first housing 210. The second housing 220 is formed into a ring shape using, for example, a member of a metal, a non-permeable synthetic resin, or the like. In the present embodiment, the second housing 220 is provided on the upper surface of the first housing 210, that is, on a surface of the first housing 210 to be positioned on a surface of the finger Fg on the back side of a hand. However, the second housing 220 may cover the entire upper surface of the first housing 210.

The first housing 210 of the housing 200 accommodates therein the battery 300 and the flexible printed circuit board 70 on which the light source 60, the first optical sensor 10A, the second optical sensor 10B, and so forth are mounted. The flexible printed circuit board 70 is accommodated in the first housing 210 with a first end 71 of the flexible printed circuit board 70 bent at a bend 73. The first end 71 bent at the bend 73 is coupled to the substrate 21 on which the first and the second optical sensors 10A and 10B are mounted. The flexible printed circuit board 70 is accommodated in the housing 200, for example, by forming the housing 200 by filling the periphery of the flexible printed circuit board 70 formed into a ring shape with a filling member in a mold. In the housing 200 of the detection device 1, the first and the second optical sensors 10A and 10B are located on the inner side with respect to the flexible printed circuit board 70 so as to be capable of receiving light from the finger Fg.

As illustrated in FIGS. 2 to 4, the flexible printed circuit board 70 extends from the first end 71 toward a second end 72 and is formed in a deformable band shape. The flexible printed circuit board 70 has a mounting area 73A, a mounting area 73B, a mounting area 73C, a mounting area 73D, and a mounting area 73E that are provided between the first end 71 and the second end 72. On a first surface 70A of the flexible printed circuit board 70, the light source 60 is mounted in the mounting area 73C located at the central area of the mounting areas 73A, 73B, 73C, 73D, and 73E; and no light source 60 is mounted in the other areas. That is, the first surface 70A of the flexible printed circuit board 70 is a surface on which the light source 60 is mounted, and a second surface 70B of the flexible printed circuit board 70 is a surface on which the light source 60 is not mounted.

On the second surface 70B on the opposite side of the first surface 70A of the flexible printed circuit board 70, electronic components are mounted in each of the mounting areas 73A, 73B, 73C, 73D, and 73E. Examples of the electronic components include, but are not limited to, a control circuit 122 in the mounting area 73A, a detection circuit 123 in the mounting area 73B, a control circuit 124 in the mounting area 73C, a control circuit 125 in the mounting area 73D, and a power supply circuit 126 in the mounting area 73E.

As illustrated in FIG. 2, on the flexible printed circuit board 70, the substrate 21 having a notch 22 illustrated in FIGS. 3 and 4 is mounted so as to straddle the vicinity of the light source 60 in the mounting area 73C. As illustrated in FIG. 3, the substrate 21 is a substrate having a front surface 21H on which the first and the second optical sensors 10A and 10B and so forth are mounted. As illustrated in FIGS. 3 and 4, the flexible printed circuit board 70 electrically couples the light source 60, the first and the second optical sensors 10A and 10B, and so forth to electronic apparatuses. The substrate 21 is coupled to the first end 71 of the flexible printed circuit board 70 so that the front surface 21H, on which the first and the second optical sensors 10A and 10B, and so forth are mounted, is continuous with the second surface 70B of the flexible printed circuit board 70.

In the present embodiment, as illustrated in FIG. 2, the first and the second optical sensors 10A and 10B are provided so as to interpose the light source 60 therebetween in a circumferential direction 200C. That is, in the detection device 1, the first optical sensor 10A, the light source 60, and the second optical sensor 10B are arranged in this order in the circumferential direction 200C. The first and the second optical sensors 10A and 10B are arranged so as to interpose the light source 60 therebetween in the circumferential direction 200C. Thereby, the light emitted by the light source 60 can be detected around the light source 60.

As illustrated in FIGS. 2 to 4, the detection device 1 includes the substrate 21 and further a terminal part 40. The substrate 21 is an insulating substrate and is formed of, for example, a film-like resin or the like and into a band shape. The substrate 21 is a deformable substrate, and the first and the second optical sensors 10A and 10B are mounted on the front surface 21H of the substrate 21. The terminal part 40 is provided at one end 21A of the front surface 21H of the substrate 21. As illustrated in FIG. 4, the substrate 21 is electrically coupled to the flexible printed circuit board 70 by being attached to the flexible printed circuit board 70 via the terminal part 40. That is, a back surface 21R of the substrate 21 is a surface continuous with the first surface 70A of the flexible printed circuit board 70 in plan view. A battery charging coil 127 is provided at the second end 72 of the flexible printed circuit board 70. The battery charging coil 127 is formed by winding a conductor in the flexible printed circuit board 70.

In the following description, a first direction Dx is one direction in a plane parallel to the substrate 21 and the flexible printed circuit board 70 and is the same direction as the circumferential direction 200C. A second direction Dy is one direction in a plane parallel to the substrate 21 and is a direction orthogonal to the first direction Dx. The second direction Dy may non-orthogonally intersect the first direction Dx. A third direction Dz is a direction orthogonal to the first direction Dx and the second direction Dy. The third direction Dz is a direction normal to the substrate 21. The term "plan view" refers to a positional relation when viewed along a direction orthogonal to the substrate 21.

As illustrated in FIGS. 2 to 4, the battery 300 is a film-type lithium-ion battery that can be curved. The battery 300 is electrically coupled to the power supply circuit 126 in the mounting area 73E on the second surface 70B of the flexible printed circuit board 70 via a coupling part 310. The width in the second direction Dy of the battery 300 is equal to or smaller than the width in the second direction Dy of the flexible printed circuit board 70, and the length in the first direction Dx of the battery 300 is smaller than the length in the first direction Dx of the flexible printed circuit board 70. This configuration allows the battery 300 of the detection device 1 to be mounted on the flexible printed circuit board 70 so as not to extend from the flexible printed circuit board 70.

As illustrated in FIGS. 5 and 6, the first end 71 of the flexible printed circuit board 70 is bent at the bend 73, so that the first and the second optical sensors 10A and 10B on the substrate 21 are respectively positioned on opposite sides of the light source 60 in the circumferential direction 200C of the housing 200. The substrate 21 has the notch 22 between opposite ends of the substrate 21 in the circumferential direction 200C of the housing 200, that is, in the first direction Dx (longitudinal direction) of the substrate 21. The first optical sensor 10A is positioned on the one end 21A side of the substrate 21 and the second optical sensor 10B is positioned on the other end 21B side of the substrate 21 with the notch 22 interposed therebetween. The flexible printed circuit board 70 is bent at the bend 73, so that the notch 22 of the substrate 21 is disposed at a location overlapping the light source 60. The placement of the notch 22 at a location overlapping the light source 60 means that the substrate 21 is attached to the flexible printed circuit board 70 with the light source 60 positioned in the area of the notch 22 of the substrate 21. The substrate 21 may be fixed to the flexible printed circuit board 70 by an adhesive member, or may not be fixed to the flexible printed circuit board 70.

The terminal part 40 is a member for electrically coupling the first and the second optical sensors 10A and 10B on the substrate 21 to the control circuit 122 and the power supply circuit 126 on the flexible printed circuit board 70. The terminal part 40 supplies power supply signals (power) from the power supply circuit 126 to the first and the second optical sensors 10A and 10B via wiring. The terminal part 40 includes a plurality of terminals (not illustrated), thus being configured to be capable of being electrically coupled to a plurality of wiring lines.

The terminal part 40 is provided at the one end 21A in the first direction Dx of the substrate 21. As illustrated in FIG. 4, the terminal part 40 is coupled to a coupling part (not illustrated) at the first end 71 of the first surface 70A of the flexible printed circuit board 70. The flexible printed circuit board 70 electrically coupled to the terminal part 40 is bent at the bend 73, so that the terminal part 40 is sandwiched between the first surfaces 70A of the flexible printed circuit board 70 as illustrated in FIG. 6, and is accommodated in the housing 200 in this state. The terminal part 40 supplies the power from the power supply circuit 126 to the first and the second optical sensors 10A and 10B.

In the present embodiment, as illustrated in FIG. 2, the flexible printed circuit board 70 is accommodated in the housing 200 such that the first surface 70A having the first and the second optical sensors 10A and 10B and the light source 60 mounted thereon faces an inner peripheral surface 200B of the housing 200. When the flexible printed circuit board 70 has a light-transmitting property, the first and the second optical sensors 10A and 10B and the light source 60 may be mounted on the second surface 70B opposite the first surface 70A. In this case, the light source 60 only needs to be disposed so as to emit light toward the flexible printed circuit board 70 and so that the light transmitted through the flexible printed circuit board 70 is emitted toward outside the housing 200.

As illustrated in FIG. 2, the light source 60 is provided in the first housing 210 of the housing 200 and is configured to be capable of emitting light toward the finger Fg wearing the housing 200. For example, an inorganic light-emitting diode (LED) or an organic electroluminescent (EL) diode (organic light-emitting diode (OLED)) is used as the light source 60. The light source 60 emits light having predetermined wavelengths. In the example illustrated in FIGS. 4 and 5, the light source 60 includes a first light source 61 that emits red light, a second light source 62 that emits near-infrared light, and a third light source 63 that emits green light.

The light emitted from the light source 60 is reflected by a surface of an object to be detected, such as the finger Fg, and enters the first and the second optical sensors 10A and 10B. Thereby, the detection device 1 can detect a fingerprint by detecting a shape of asperities on the surface of the finger Fg or the like. Alternatively, the light emitted from the light source 60 may be reflected in the finger Fg or the like, or transmitted through the finger Fg or the like and enter the first and the second optical sensors 10A and 10B. Thereby, the detection device 1 can detect the information on the living body in the finger Fg or the like. Examples of the information on the living body include, but are not limited to, pulse waves, pulsation, and a vascular image of the finger or a palm. That is, the detection device 1 may be configured as a fingerprint detection device that detects the fingerprint or a vein detection device that detects a pattern of blood vessels such as veins.

Each of the first and the second optical sensors 10A and 10B detects the light emitted by the light source 60 and reflected by the finger Fg, light directly incident on the optical sensor, and other light. The first and the second optical sensors 10A and 10B are each an organic photodiode (OPD). The first optical sensor 10A is provided in the housing 200 so as to be adjacent to one end of the light source 60 in the circumferential direction 200C of the housing 200. The second optical sensor 10B is provided in the housing 200 so as to be adjacent to the other end of the light source 60 in the circumferential direction 200C of the housing 200.

As illustrated in FIG. 7, the first optical sensor 10A includes the substrate 21 and a photodiode PD. In the present embodiment, the first optical sensor 10A further includes wiring lines 26 and an insulating layer 27.

The wiring lines 26 are provided on the upper surface of an area of the substrate 21 corresponding to the first optical sensor 10A. The wiring lines 26 are each a shield layer formed of, for example, metal wiring, and is formed of a material having better conductivity than a lower electrode 11 of the photodiode PD. The wiring lines 26 are provided between the substrate 21 and the photodiode PD in the third direction Dz. The wiring lines 26 are electrically coupled to the terminal part 40 on the substrate 21. The wiring lines 26 may be formed in, for example, the same layer as the lower electrode 11, or may be formed of metal. The insulating layer 27 is provided on the substrate 21 so as to cover the wiring lines 26. The insulating layer 27 may be an inorganic insulating film or an organic insulating film.

The photodiode PD is provided on the insulating layer 27. The photodiode PD includes the lower electrode 11, a lower buffer layer 12, an active layer 13, an upper buffer layer 14, and an upper electrode 15 (upper electrode 15A). In the photodiode PD, the lower electrode 11, the lower buffer layer 12 (hole transport layer), the active layer 13, the upper buffer layer 14 (electron transport layer), and the upper electrode 15A are stacked in this order in the third direction Dz orthogonal to the substrate 21.

The lower electrode 11 is an anode electrode of the photodiode PD and is formed of a light-transmitting conductive material such as indium tin oxide (ITO), for example. The active layer 13 changes in characteristics (such as voltage-current characteristics and resistance value) depending on light emitted thereto. An organic material is used as a material of the active layer 13. Specifically, the active layer 13 has a bulk heterostructure containing a mixture of a p-type organic semiconductor and an n-type fullerene derivative ((6,6)-phenyl-C₆₁-butyric acid methyl ester (PCBM)) that is an n-type organic semiconductor. As the active layer 13, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 13 can be formed by a vapor deposition process (dry process) using any of the low-molecular-weight organic materials listed above. In this case, the active layer 13 may be, for example, a multilayered film of CuPc and F₁₆CuPc, or a multilayered film of rubrene and C₆₀. The active layer 13 can also be formed by a coating process (wet process). In this case, the active layer 13 is made using a material obtained by combining any of the above-listed low-molecular-weight organic materials with a high-molecular-weight organic material. As the high-molecular-weight organic material, for example, poly(3-hexylthiophene) (P3HT) and F8-alt-benzothiadiazole (F8BT) can be used. The active layer 13 can be a film made of a mixture of P3HT and PCBM, or a film made of a mixture of F8BT and PDI.

The lower buffer layer 12 is a hole transport layer. The upper buffer layer 14 is an electron transport layer. The lower buffer layer 12 and the upper buffer layer 14 are provided to facilitate holes and electrons generated in the active layer 13 to reach the lower electrode 11 or the upper electrode 15. The lower buffer layer 12 (hole transport layer) is in direct contact with the top of the lower electrode 11 and is also provided in an area between the adjacent lower electrodes 11. The active layer 13 is in direct contact with the top of the lower buffer layer 12. The material of the hole transport layer is a metal oxide layer. Tungsten oxide (WO₃), molybdenum oxide, or the like is used as the metal oxide layer.

The upper buffer layer 14 (electron transport layer) is in direct contact with the top of the active layer 13, and the upper electrode 15 is in direct contact with the top of the upper buffer layer 14. Polyethylenimine ethoxylated (PEIE) is used as a material of the electron transport layer.

The materials and the manufacturing methods of the lower buffer layer 12, the active layer 13, and the upper buffer layer 14 are merely exemplary, and other materials and manufacturing methods may be used. For example, each of the lower buffer layer 12 and the upper buffer layer 14 is not limited to a single-layer film, and may be formed as a multilayered film that includes an electron blocking layer and a hole blocking layer.

The upper electrode 15 is provided on the upper buffer layer 14. The upper electrode 15 is a cathode electrode of the photodiode PD and is continuously formed over the entire first and second optical sensors 10A and 10B. In other words, the upper electrode 15 is continuously provided on a plurality of the photodiodes PD. The upper electrode 15 faces the lower electrode 11 with the lower buffer layer 12, the active layer 13, and the upper buffer layer 14 interposed therebetween. The upper electrode 15 is formed of, for example, a light-transmitting conductive material such as ITO or indium zinc oxide (IZO). A part of an end of an upper surface 150 of the upper electrode 15 is electrically coupled to a conductor 24 and electrically coupled to a power supply electrode 211 via the conductor 24. The power supply electrode 211 is electrically coupled to the battery 300, thus being configured to be capable of being supplied with power from the battery 300. A sealing film 160 is provided on the upper electrode 15 and so forth of the first optical sensor 10A. An inorganic film, such as a silicon nitride film or an aluminum oxide film, or a resin film, such as an acrylic film, is used as the sealing film 160. The sealing film 160 is not limited to a single layer and may be a multilayered film having two or more layers obtained by combining the inorganic film with the resin film mentioned above. In the detection device 1, the sealing film 160 well seals the photodiode PD and thus can reduce moisture entering the photodiode PD from the upper surface side thereof.

As illustrated in FIG. 8, the second optical sensor 10B includes the lower electrode 11 of the second optical sensor 10B in an area of the substrate 21 different from the area for the lower electrode 11 of the first optical sensor 10A. The lower electrode 11 is covered with the lower buffer layer 12, the active layer 13, the upper buffer layer 14, and the upper electrode 15 (upper electrode 15B). In the present embodiment, the second optical sensor 10B includes the substrate 21, the photodiode PD, the wiring lines 26, and the insulating layer 27. The photodiode PD, the wiring lines 26, and the insulating layer 27 have the same configuration as that of the photodiode PD, the wiring lines 26, and the insulating layer 27 of the first optical sensor 10A described above. That is, the photodiode PD of the second optical sensor 10B includes the lower electrode 11, the lower buffer layer 12, the active layer 13, the upper buffer layer 14, and the upper electrode 15B. In the second optical sensor 10B, the photodiode PD is well sealed by providing the sealing film 160 on the upper electrode 15B and so forth.

As illustrated in FIG. 4, the substrate 21 has the areas of the first and the second optical sensors 10A and 10B, and is integrally formed in one common substrate. The substrate 21 has the notch 22 formed between the area for the first optical sensor 10A and the area for the second optical sensor 10B in the first direction Dx. The substrate 21 has the notch 22 and a joint 23, the notch 22 is provided between the first and the second optical sensors 10A and 10B, and the joint 23 is adjacent to the notch 22 and lies between the first and the second optical sensors 10A and 10B.

The notch 22 is formed to have a longer length than the length of the light source 60 in the first direction Dx. The notch 22 is formed to have a longer length than the length of the light source 60 and a shorter distance than the length (width) the substrate 21 in the second direction Dy. The substrate 21 is integrally formed by connecting the first optical sensor 10A to the second optical sensor 10B via the joint 23 along the notch 22. The lower buffer layer 12, the active layer 13, the upper buffer layer 14, and an electrode connector 151 of the upper electrode 15 are arranged at the joint 23. With this configuration, the joint 23 integrally forms the upper electrodes 15A and 15B of the first and the second optical sensors 10A and 10B. The first and the second optical sensors 10A and 10B are connected by the joint 23 and operated by power supplied from the power supply electrode 211 to the common upper electrode 15.

The notch 22 is formed in a shape that allows the light source 60 to be located therein. In the present embodiment, the notch 22 is formed into a substantially rectangular shape in plan view, but may have a semicircular, triangular, polygonal, or other shape, for example. The notch 22 may be a through-hole capable of transmitting light from the light source 60. The electrode connector 151 is provided on the joint 23 of the substrate 21 so as to be stacked on the upper buffer layer 14, the active layer 13, and the lower buffer layer 12.

The wiring lines 26 of the substrate 21 are coupled to the control circuit 122 via a plurality of signal lines (not illustrated) of the flexible printed circuit board 70. In other words, the control circuit 122 is electrically coupled to the lower electrodes 11 of the first and the second optical sensors 10A and 10B via the signal lines.

The control circuit 122 is a circuit that controls detection operations by supplying control signals to the photodiodes PD. Each of the photodiodes PD outputs an electrical signal in response to light emitted thereto as a detection signal Vdet to the detection circuit 123. The detection circuit 123 is a detection circuit for the detection signal Vdet. The detection circuit 123 is an analog front-end (AFE) circuit, for example. The detection circuit 123 is a signal processing circuit having functions of at least a detection signal amplifying circuit and an analog-to-digital (A/D) conversion circuit. The detection signal amplifying circuit amplifies the detection signal Vdet. The A/D conversion circuit converts analog signals output from the detection signal amplifying circuit into digital signals.

The control circuit 124 for charging controls power transferred wirelessly to the battery charging coil 127 from an external source using an electromagnetic method. One of an electromagnetic induction method, an electromagnetic resonance method, a radio wave method, and the like is selected as a method of coupling for the wireless power transmission. The control circuit 124 charges the battery 300 using the power transmitted by the wireless power transmission.

The control circuit 125 supplies a control signal to the light source 60 to control lighting and non-lighting of the light source 60. The power supply circuit 126 supplies power supply signals (power) from the battery 300 to the first and the second optical sensors 10A and 10B via the terminal part 40.

The configuration example of the detection device 1 according to the present embodiment has been described above. The configuration described above using FIGS. 1 to 8 is merely an example, and the configuration of the detection device 1 according to the present embodiment is not limited to the example. The configuration of the detection device 1 according to the present embodiment can be flexibly modified depending on the specification or operation.

### Assembly Example of Detection Device

The following describes an assembly example of the detection device 1. As illustrated in FIGS. 3 and 4, the light source 60 is mounted on the first surface 70A of the flexible printed circuit board 70; and the control circuit 122, the detection circuit 123, the control circuit 124, the control circuit 125, and the power supply circuit 126 are mounted on the second surface 70B of the flexible printed circuit board 70. The first and the second optical sensors 10A and 10B are formed on the substrate 21, and the terminal part 40 is mounted on the substrate 21. The coupling part on the first surface 70A of the flexible printed circuit board 70 is coupled to the terminal part 40, so that the substrate 21 is coupled to the first end 71 of the flexible printed circuit board 70. As illustrated in FIG. 5, the flexible printed circuit board 70 is bent at the bend 73, and while bringing the other end 21B of the substrate 21 closer to the first surface 70A of the flexible printed circuit board 70, the substrate 21 is disposed on the first surface 70A of the flexible printed circuit board 70 so that the notch 22 of the substrate 21 overlaps the light source 60. The battery 300 is electrically coupled to the flexible printed circuit board 70 at the second end 72. The substrate 21, the flexible printed circuit board 70, and the battery 300 are formed into a ring shape and accommodated in a mold, and the periphery thereof is filled with a filling member to form the housing 200, whereby the substrate 21, the flexible printed circuit board 70, and the battery 300 are accommodated in the housing 200. Thus, as illustrated in FIG. 2, the detection device 1 is formed as a device in which the bent flexible printed circuit board 70, the substrate 21 with the notch 22 overlapping the light source 60, and the battery 300 are accommodated in the housing 200.

### Operation Example of Detection Device

The following describes a detection example of the detection device 1 worn on the finger Fg. In the example illustrated in FIG. 2, the detection device 1 is in a state where the inner peripheral surface 200B of the first housing 210 of the housing 200 is in contact with or in proximity to the finger Fg. The detection device 1 operates the first and the second optical sensors 10A and 10B by supplying the power from the battery 300 to the common upper electrode 15 via the terminal part 40. The detection device 1 turns on the light source 60 so that the light source 60 emits light toward the finger Fg. The light source 60 emits the light toward one side and the other side in the circumferential direction 200C. In the detection device 1, the first and the second optical sensors 10A and 10B receive light reflected by the finger Fg or the like. The detection device 1 detects the information on the living body in the finger Fg based on the amount of light detected by each of the two photodiodes PD of the first and the second optical sensors 10A and 10B.

Thus, in the detection device 1, the substrate 21 including the first and the second optical sensors 10A and 10B is coupled to the flexible printed circuit board 70 via the terminal part 40, and the flexible printed circuit board 70 is bent, so that the notch 22 of the substrate 21 is located in the position overlapping the light source 60 on the flexible printed circuit board 70. With this configuration, in the detection device 1, even when the substrate 21 and the flexible printed circuit board 70 including the light source 60 that are separate members are coupled in series, the substrate 21 can be disposed by bending the flexible printed circuit board 70 in the coupling direction. Therefore, the accommodation space can be downsized. As a result, the detection device 1 can prevent an increase in size of a member in which the substrate 21 including the multiple optical sensors and the flexible printed circuit board 70 including the light source 60 are coupled and accommodated.

In the detection device 1, the first and the second optical sensors 10A and 10B can share the lower buffer layer 12, the active layer 13, the upper buffer layer 14, and the upper electrode 15. With this configuration, the detection device 1 only needs to supply power to the integrated upper electrode 15 for the first and the second optical sensors 10A and 10B. Therefore, the configuration of the substrate 21 can be simplified. As a result, an increase in size of the substrate 21 of the detection device 1 can be inhibited even when the multiple optical sensors are arranged on the substrate 21.

In the detection device 1, the electronic components are mounted on the second surface 70B of the flexible printed circuit board 70 on the opposite side of the light source 60. Thus, in the detection device 1, the electronic components is mounted on the second surface 70B of the flexible printed circuit board 70, and the light source 60 is mounted on the first surface 70A on the opposite side of the second surface 70B, whereby the configuration of the flexible printed circuit board 70 is simplified, and the accommodation space is further downsized.

In the detection device 1, the substrate 21 and the flexible printed circuit board 70 are accommodated in the ring-shaped housing 200. This configuration allows the detection device 1 to accurately detect the light emitted from the light source 60 in a wide area of the housing 200 using the multiple optical sensors without increasing the size of the ring-shaped housing 200.

### Second Embodiment

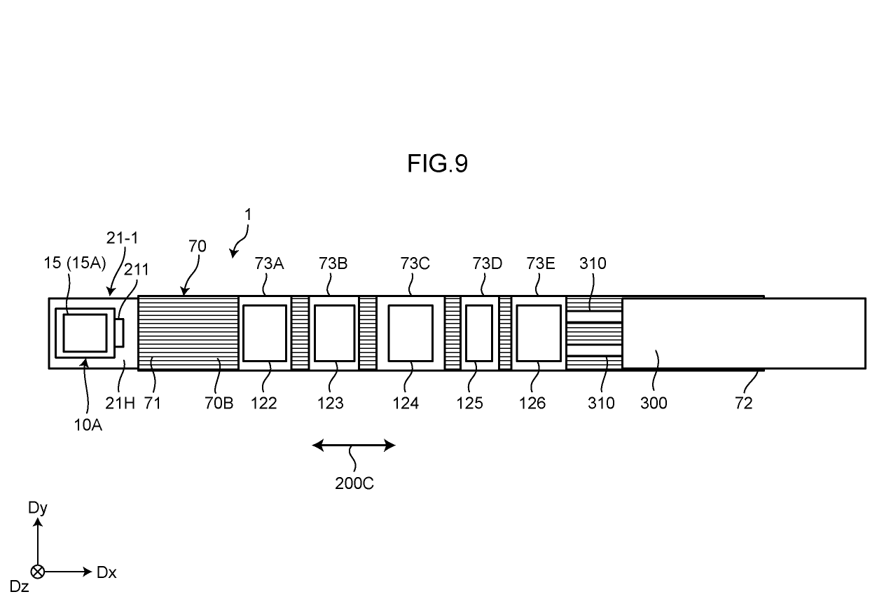

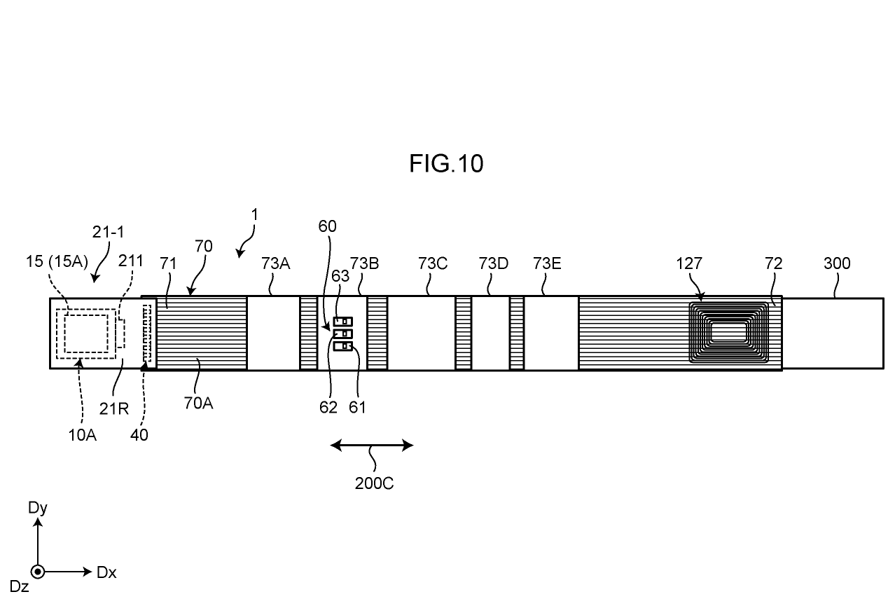

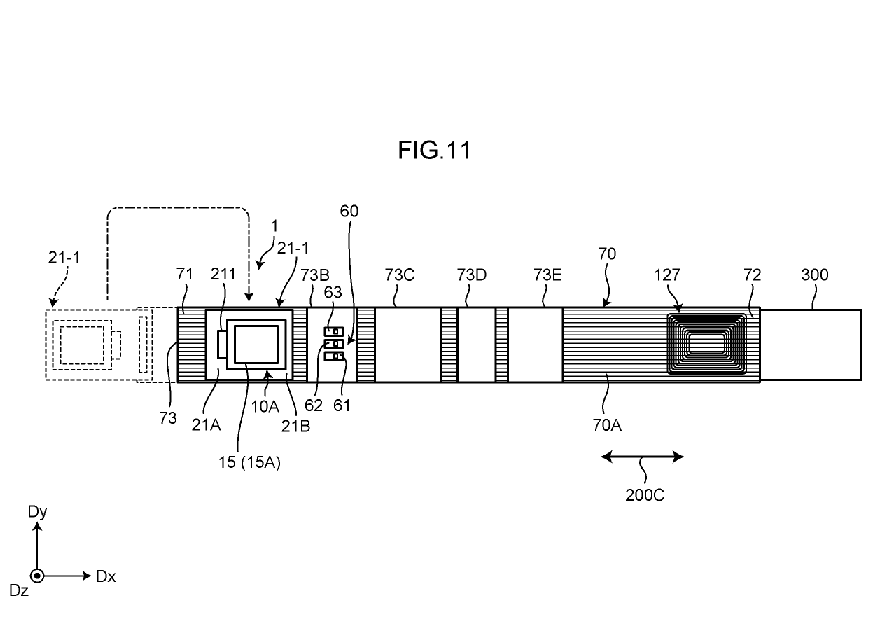

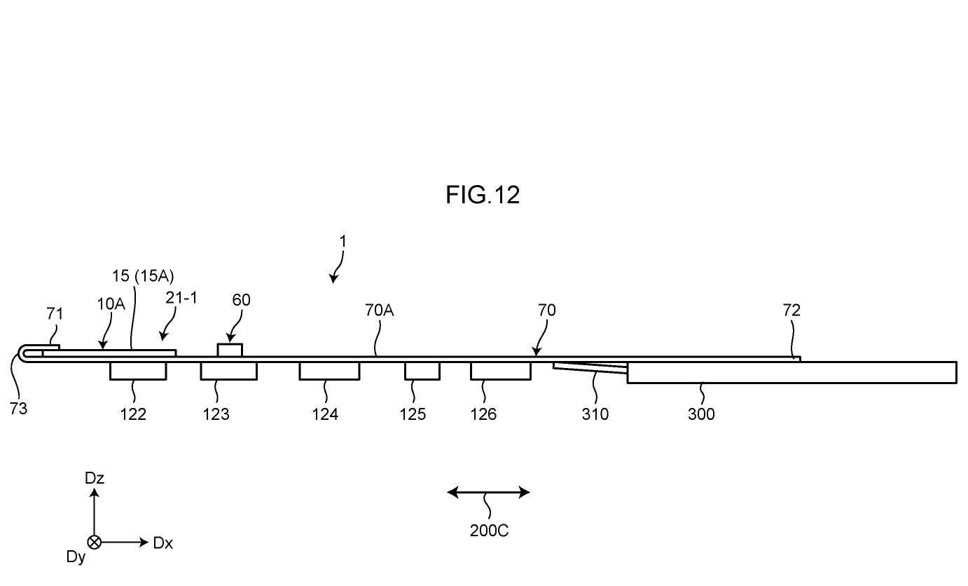

FIG. 9 is a development view illustrating an exemplary development of the front surface side of a substrate and the flexible printed circuit board of a second embodiment. FIG. 10 is a development view illustrating an exemplary development of the back surface side of the substrate and the flexible printed circuit board of the second embodiment. FIG. 11 is a development view illustrating a state where the flexible printed circuit board illustrated in FIG. 10 is bent. FIG. 12 is a side view of the substrate and the flexible printed circuit board illustrated in FIG. 11.

In the second embodiment, the detection device 1 includes the housing 200, the terminal part 40, the light source 60, the first optical sensor 10A, and the flexible printed circuit board 70 that have been described above, and a substrate 21-1. The detection device 1 is a device that includes the battery 300 accommodated in the housing 200 and coupled to the flexible printed circuit board 70, and is operated by the power of the battery 300. The multiple electronic components are mounted on the flexible printed circuit board 70. The electronic components include the control circuit 122 in the mounting area 73A, the detection circuit 123 in the mounting area 73B, the control circuit 124 in the mounting area 73C, the control circuit 125 in the mounting area 73D, and the power supply circuit 126 in the mounting area 73E that have been described above. In the second embodiment, the detection device 1 accommodates the terminal part 40, the light source 60, the first optical sensor 10A, the flexible printed circuit board 70, and the substrate 21-1 in the ring-shaped housing 200 in same way as in the first embodiment.

The substrate 21-1 is an insulating substrate and is formed of, for example, a film-like resin or the like and formed into a band shape. The substrate 21-1 is a deformable substrate, and the first optical sensor 10A is mounted on the front surface 21H of the substrate 21-1 near the other end 21B. The terminal part 40 is provided at the one end 21A of the front surface 21H of the substrate 21-1. As illustrated in FIG. 10, the substrate 21-1 is electrically coupled to the flexible printed circuit board 70 by being attached to the flexible printed circuit board 70 via the terminal part 40. That is, the back surface 21R of the substrate 21-1 is a surface continuous with the first surface 70A of the flexible printed circuit board 70 in plan view.

The substrate 21-1 is provided with a smaller number of the optical sensors and has a smaller length in the first direction Dx than in the first embodiment. For this reason, the flexible printed circuit board 70 differs from that of the first embodiment in that the light source 60 is mounted not in the mounting area 73C but in the mounting area 73B on the first surface 70A based on the length of the substrate 21-1, the length of the bent part, and so forth. In the second embodiment, on the flexible printed circuit board 70, the position of the light source 60 is shifted toward the first end 71 because the length in the first direction Dx (circumferential direction 200C) of the substrate 21 has changed to that of the substrate 21-1.

The first end 71 of the flexible printed circuit board 70 is bent at the bend 73, whereby the first optical sensor 10A on the substrate 21-1 illustrated in FIGS. 9 and 10 is positioned at a location adjacent to the light source 60 in the circumferential direction 200C of the housing 200. As illustrated in FIG. 11, the flexible printed circuit board 70 is bent at the bend 73, whereby the substrate 21-1 is located near the light source 60. The substrate 21-1 illustrated in FIG. 12 may be fixed to the flexible printed circuit board 70 by an adhesive member, or may not be fixed to the flexible printed circuit board 70.

The terminal part 40 illustrated in FIG. 10 is a member for electrically coupling the first optical sensor 10A on the substrate 21-1 to the control circuit 122 and the power supply circuit 126 on the flexible printed circuit board 70. The terminal part 40 supplies a power supply signal (power) from the power supply circuit 126 to the first optical sensor 10A via wiring. The terminal part 40 includes a plurality of terminals (not illustrated), thus being configured to be capable of being electrically coupled to a plurality of wiring lines.

The flexible printed circuit board 70 illustrated in FIG. 12 is accommodated in the housing 200 such that the first surface 70A having the first optical sensor 10A and the light source 60 mounted thereon faces the inner peripheral surface 200B of the housing 200. When the flexible printed circuit board 70 has a light-transmitting property, the first optical sensor 10A and the light source 60 may be mounted on the second surface 70B opposite the first surface 70A. In this case, the light source 60 only needs to be disposed so as to emit light toward the flexible printed circuit board 70 and so that the light transmitted through the flexible printed circuit board 70 is emitted toward outside the housing 200.

The light emitted from the light source 60 is reflected by the surface of the object to be detected, such as the finger Fg, and enters the first optical sensor 10A. Thereby, the detection device 1 can detect the fingerprint by detecting the shape of the asperities on the surface of the finger Fg or the like. Alternatively, the light emitted from the light source 60 may be reflected in the finger Fg or the like, or transmitted through the finger Fg or the like and enter the first optical sensor 10A. Thereby, the detection device 1 can detect the information on the living body in the finger Fg or the like. Examples of the information on the living body include, but are not limited to, the pulse waves, the pulsation, and the vascular image of the finger or the palm. That is, the detection device 1 may be configured as the fingerprint detection device that detects the fingerprint or the vein detection device that detects the pattern of the blood vessels such as the veins.

The first optical sensor 10A detects the light emitted by the light source 60 and reflected by the finger Fg, light directly incident on the optical sensor, and other light. The first optical sensor 10A is an organic photodiode (OPD). The first optical sensor 10A is provided in the housing 200 so as to be adjacent to one end of the light source 60 in the circumferential direction 200C of the housing 200.

The configuration example of the detection device 1 according to the second embodiment has been described above. The configuration described above using FIGS. 9 to 12 is merely an example, and the configuration of the detection device 1 according to the second embodiment is not limited to the example. The configuration of the detection device 1 according to the second embodiment can be flexibly modified depending on the specification or operation.

### Assembly Example of Detection Device

The following describes an assembly example of the detection device 1 according to the second embodiment. As illustrated in FIGS. 9 and 10, the light source 60 is mounted on the first surface 70A of the flexible printed circuit board 70; and the control circuit 122, the detection circuit 123, the control circuit 124, the control circuit 125, and the power supply circuit 126 are mounted on the second surface 70B of the flexible printed circuit board 70. The first optical sensor 10A is formed on the substrate 21-1, and the terminal part 40 is mounted on the substrate 21-1. The coupling part on the first surface 70A of the flexible printed circuit board 70 is coupled to the terminal part 40, so that the substrate 21-1 is coupled to the first end 71 of the flexible printed circuit board 70. As illustrated in FIG. 11, the flexible printed circuit board 70 is bent at the bend 73, and while bringing the other end 21B of the substrate 21-1 closer to the light source 60, the substrate 21-1 is disposed on the first surface 70A of the flexible printed circuit board 70 so that the first optical sensor 10A on the substrate 21-1 is located adjacent to the light source 60. In the example illustrated in FIG. 11, the substrate 21-1 is disposed so as to cover the mounting area 73A on the first surface 70A of the flexible printed circuit board 70 and so that the other end 21B is located adjacent to the light source 60. The battery 300 is electrically coupled to the flexible printed circuit board 70 at the second end 72. The substrate 21-1, the flexible printed circuit board 70, and the battery 300 are formed into a ring shape and accommodated in a mold, and the periphery thereof is filled with a filling member to form the housing 200, whereby the substrate 21-1, the flexible printed circuit board 70, and the battery 300 are accommodated in the housing 200. Thus, the detection device 1 is formed as a device in which the bent flexible printed circuit board 70, the substrate 21-1 with the first optical sensor 10A located adjacent to the light source 60, and the battery 300 are accommodated in the housing 200.

### Operation Example of Detection Device

The following describes a detection example of the detection device 1 worn on the finger Fg. The detection device 1 is in the state where the inner peripheral surface 200B of the first housing 210 of the housing 200 is in contact with or in proximity to the finger Fg. The detection device 1 operates the first optical sensor 10A by supplying the power from the battery 300 to the upper electrode 15 of the first optical sensor 10A via the terminal part 40. The detection device 1 turns on the light source 60 so that the light source 60 emits the light toward the finger Fg. The light source 60 emits the light toward the one side and the other side in the circumferential direction 200C. In the detection device 1, the first optical sensor 10A receives the light reflected by the finger Fg or the like. The detection device 1 detects the information on the living body in the finger Fg based on the amount of the light detected by each of the photodiodes PD of the first optical sensor 10A.

Thus, in the detection device 1, the substrate 21-1 including the first optical sensor 10A is coupled to the flexible printed circuit board 70 via the terminal part 40, and the flexible printed circuit board 70 is bent, so that the first optical sensor 10A on the substrate 21-1 is located in a position adjacent to the light source 60 on the flexible printed circuit board 70. With this configuration, in the detection device 1, even when the substrate 21-1 and the flexible printed circuit board 70 including the light source 60 that are separate members are coupled in series, the substrate 21-1 can be disposed by bending the flexible printed circuit board 70. Therefore, the accommodation space can be downsized. As a result, in the detection device 1, the multiple electronic components can be easily mounted on the flexible printed circuit board 70, and the flexibility of arrangement of the first optical sensor 10A and the light source 60 can be improved. Even when the multiple electronic components are mounted on the flexible printed circuit board 70, the detection device 1 can accommodate the first optical sensor 10A and the light source 60 in the state of being adjacent to each other without increasing the size of the accommodating housing 200.

### Third Embodiment

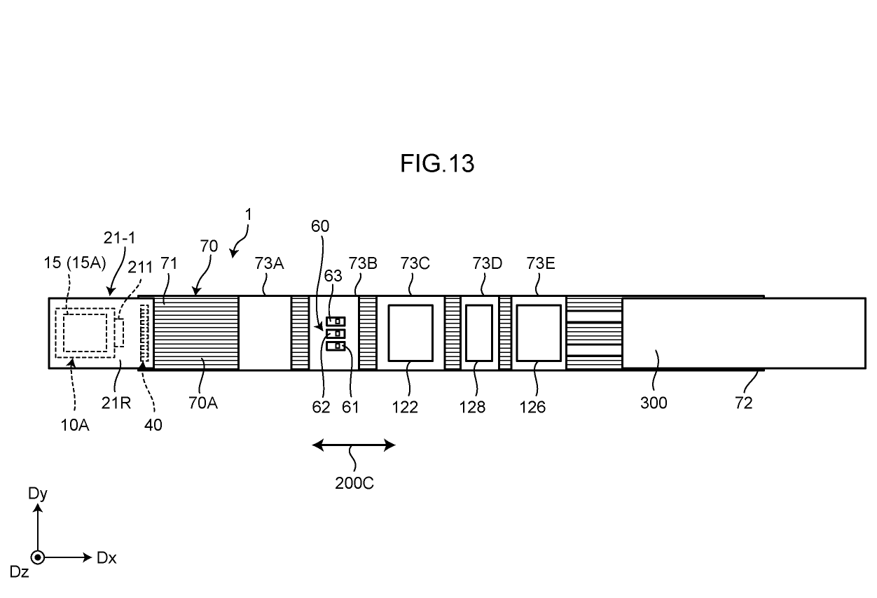

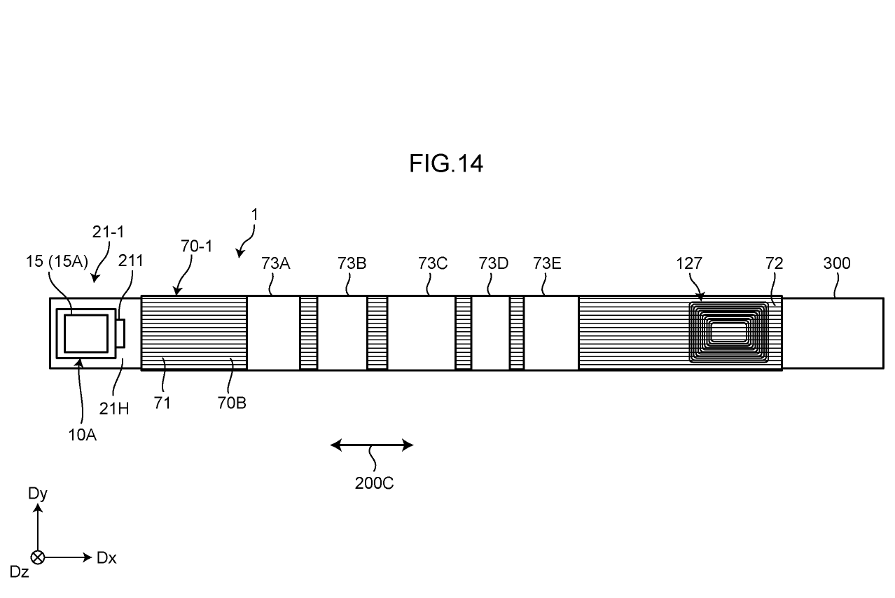

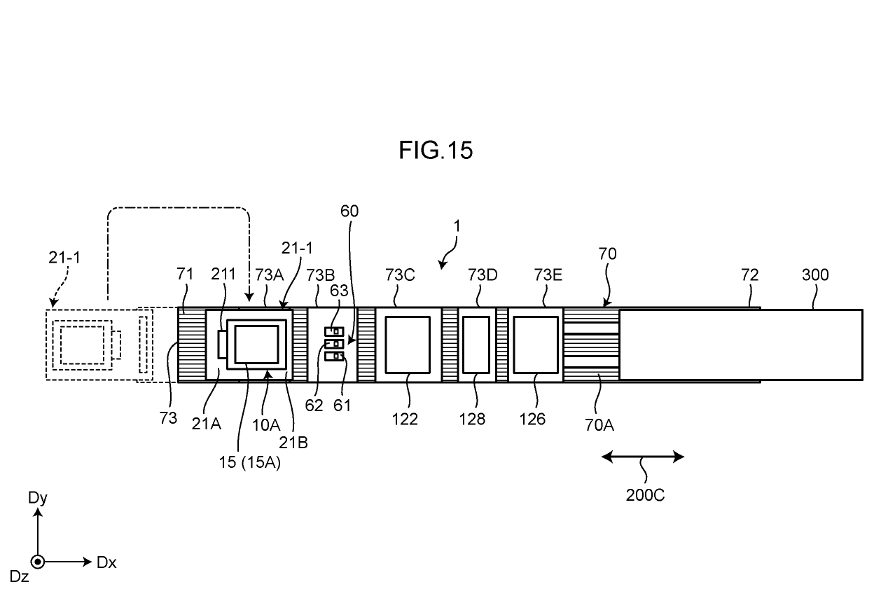

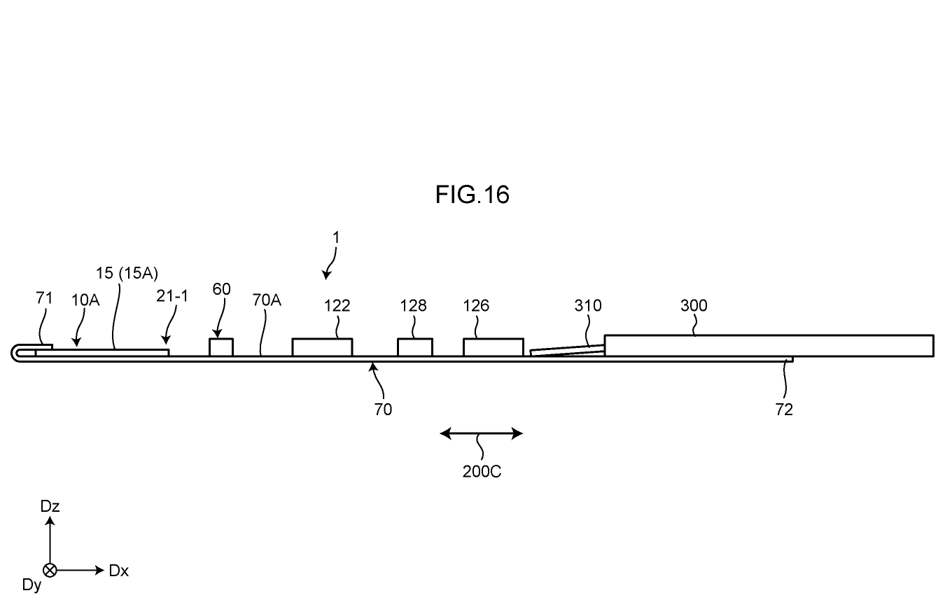

FIG. 13 is a development view illustrating an exemplary development of the front surface side of the substrate and the flexible printed circuit board of a third embodiment. FIG. 14 is a development view illustrating an exemplary development of the back surface side of the substrate and the flexible printed circuit board of the third embodiment. FIG. 15 is a development view illustrating a state where the flexible printed circuit board illustrated in FIG. 14 is bent. FIG. 16 is a side view of the substrate and the flexible printed circuit board illustrated in FIG. 15.

In the third embodiment, the detection device 1 includes the housing 200, the substrate 21-1, the terminal part 40, the light source 60, the first optical sensor 10A, and the flexible printed circuit board 70 that have been described above. The detection device 1 is a device that includes the battery 300 accommodated in the housing 200 and coupled to the flexible printed circuit board 70, and is operated by the power of the battery 300. The first optical sensor 10A and the substrate 21-1 have the same configurations as those of the first optical sensor 10A and the substrate 21-1 in the second embodiment. The multiple electronic components are mounted on the flexible printed circuit board 70. The electronic components include the control circuit 122 and the power supply circuit 126 described above, and a control circuit 128. In the third embodiment, the detection device 1 accommodates the terminal part 40, the light source 60, the first optical sensor 10A, the flexible printed circuit board 70, and the substrate 21-1 in the ring-shaped housing 200 in same way as in the first embodiment and the second embodiment.

In the third embodiment, as illustrated in FIGS. 13 and 14, the flexible printed circuit board 70 has the first surface 70A and the second surface 70B; the control circuit 122, the power supply circuit 126, and the control circuit 128 are mounted on the first surface 70A on which the light source 60 is mounted, while no electronic components are mounted on the second surface 70B. On the first surface 70A of the flexible printed circuit board 70, nothing is mounted in the mounting area 73A, whereas the light source 60, the control circuit 122, the control circuit 128, and the power supply circuit 126 are mounted in the mounting areas 73B, 73C, 73D, and 73E, respectively. The control circuit 128 includes one circuit obtained by combining the detection circuit 123, the control circuit 124, and the control circuit 125 that have been described above.

The first end 71 of the flexible printed circuit board 70 is bent at the bend 73, whereby the first optical sensor 10A on the substrate 21-1 illustrated in FIGS. 13 and 14 is positioned at a location adjacent to the light source 60 in the circumferential direction 200C of the housing 200 while bringing the other end 21B of the substrate 21-1 closer to the light source 60. As illustrated in FIG. 15, the flexible printed circuit board 70 is bent at the bend 73, whereby the substrate 21-1 is located near the light source 60. The substrate 21-1 illustrated in FIG. 16 may be fixed to the flexible printed circuit board 70 by an adhesive member, or may not be fixed to the flexible printed circuit board 70.

The flexible printed circuit board 70 illustrated in FIG. 16 is accommodated in the housing 200 such that the first surface 70A having the first optical sensor 10A, the light source 60, and the electronic components mounted thereon faces the inner peripheral surface 200B of the housing 200. When the flexible printed circuit board 70 has a light-transmitting property, the first optical sensor 10A, the light source 60, and the electronic components may be mounted on the second surface 70B opposite the first surface 70A. In this case, the light source 60 only needs to be disposed so as to emit light toward the flexible printed circuit board 70 and so that the light transmitted through the flexible printed circuit board 70 is emitted toward outside the housing 200.

The light emitted from the light source 60 is reflected by the surface of the object to be detected, such as the finger Fg, and enters the first optical sensor 10A. Thereby, the detection device 1 can detect the fingerprint by detecting the shape of the asperities on the surface of the finger Fg or the like. Alternatively, the light emitted from the light source 60 may be reflected in the finger Fg or the like, or transmitted through the finger Fg or the like and enter the first optical sensor 10A. Thereby, the detection device 1 can detect the information on the living body in the finger Fg or the like. Examples of the information on the living body include, but are not limited to, the pulse waves, the pulsation, and the vascular image of the finger or the palm. That is, the detection device 1 may be configured as the fingerprint detection device that detects the fingerprint or the vein detection device that detects the pattern of the blood vessels such as the veins.

The configuration example of the detection device 1 according to the third embodiment has been described above. The configuration described above using FIGS. 13 to 16 is merely an example, and the configuration of the detection device 1 according to the third embodiment is not limited to the example. The configuration of the detection device 1 according to the third embodiment can be flexibly modified depending on the specification or operation.

### Assembly Example of Detection Device

The following describes an assembly example of the detection device 1 according to the third embodiment. As illustrated in FIGS. 13 and 14, the light source 60, the control circuit 122, the control circuit 128, and the power supply circuit 126 are mounted on the first surface 70A of the flexible printed circuit board 70. The first optical sensor 10A is formed on the substrate 21-1, and the terminal part 40 is mounted on the substrate 21-1. The coupling part on the first surface 70A of the flexible printed circuit board 70 is coupled to the terminal part 40, so that the substrate 21-1 is coupled to the first end 71 of the flexible printed circuit board 70. As illustrated in FIG. 15, the flexible printed circuit board 70 is bent at the bend 73, and the substrate 21-1 is disposed on the first surface 70A of the flexible printed circuit board 70 so that the first optical sensor 10A on the substrate 21-1 is located adjacent to the light source 60. In the example illustrated in FIG. 15, the substrate 21-1 is disposed so as to cover the mounting area 73A on the first surface 70A of the flexible printed circuit board 70 and so that the other end 21B is located adjacent to the light source 60. The battery 300 is electrically coupled to the flexible printed circuit board 70 at the second end 72. The substrate 21-1, the flexible printed circuit board 70, and the battery 300 are formed into a ring shape and accommodated in a mold, and the periphery thereof is filled with a filling member to form the housing 200, whereby the substrate 21-1, the flexible printed circuit board 70, and the battery 300 are accommodated in the housing 200. Thus, the detection device 1 is formed as a device in which the bent flexible printed circuit board 70, the substrate 21-1 with the first optical sensor 10A located adjacent to the light source 60, and the battery 300 are accommodated in the housing 200.

### Operation Example of Detection Device

The following describes a detection example of the detection device 1 worn on the finger Fg. The detection device 1 is in the state where the inner peripheral surface 200B of the first housing 210 of the housing 200 is in contact with or in proximity to the finger Fg. The detection device 1 operates the first optical sensor 10A by supplying the power from the battery 300 to the upper electrode 15 of the first optical sensor 10A via the terminal part 40. The detection device 1 turns on the light source 60 so that the light source 60 emits the light toward the finger Fg. The light source 60 emits the light toward the one side and the other side in the circumferential direction 200C. In the detection device 1, the first optical sensor 10A receives the light reflected by the finger Fg or the like. The detection device 1 detects the information on the living body in the finger Fg based on the amount of the light detected by each of the photodiodes PD of the first optical sensor 10A.

Thus, in the detection device 1, the substrate 21-1 including the first optical sensor 10A is coupled to the flexible printed circuit board 70 via the terminal part 40, and the flexible printed circuit board 70 is bent, so that the first optical sensor 10A on the substrate 21-1 is located in a position adjacent to the light source 60 on the flexible printed circuit board 70. With this configuration, in the detection device 1, even when the substrate 21-1 and the flexible printed circuit board 70 including the light source 60 that are separate members are coupled in series, the substrate 21-1 can be disposed by bending the flexible printed circuit board 70. Therefore, the accommodation space can be downsized. As a result, in the detection device 1, the multiple electronic components and the light source 60 can be easily mounted on the flexible printed circuit board 70, and the flexibility of arrangement of the first optical sensor 10A and the light source 60 can be improved. Even when the multiple electronic components are mounted on the flexible printed circuit board 70, the detection device 1 can accommodate the first optical sensor 10A and the light source 60 in the state of being adjacent to each other without increasing the size of the accommodating housing 200.

In the detection device 1, the multiple electronic components are mounted on the first surface 70A of the flexible printed circuit board 70 on which the light source 60 is mounted. Thus, in the detection device 1, the light source 60 and the electronic components are mounted on the first surface 70A of the flexible printed circuit board 70, whereby the mounting of the flexible printed circuit board 70 is further simplified.

In each of the embodiments described above, the case has been described where, in the detection device 1, the substrate 21 or 21-1, the flexible printed circuit board 70, the battery 300, and so forth are accommodated in the ring-shaped housing 200, but the present disclosure is not limited to this case. The detection device 1 may, for example, be configured such that those components are accommodated in a square housing or are attached to the object to be measured without being accommodated in a housing.

The components in the embodiments described above can be combined as appropriate. Other operational advantages accruing from the aspects described in the embodiments of the present disclosure that are obvious from the description herein, or that are conceivable as appropriate by those skilled in the art will naturally be understood as accruing from the present disclosure.

What is claimed is:

1. A detection device comprising:
a substrate having opposite ends in a first direction with a notch interposed therebetween;
a terminal part provided at one end in the first direction of the substrate;
a first optical sensor provided on the substrate between the notch and the terminal part;
a second optical sensor provided on the substrate between the notch and another end of the substrate; and
a flexible printed circuit board on which a light source and a plurality of electronic components are mounted, wherein
a lower electrode, a lower buffer layer, an active layer, an upper buffer layer, an upper electrode, and a sealing film are stacked on the substrate in the order as listed, in each of the first optical sensor and the second optical sensor,
the lower electrodes of the first optical sensor and the second optical sensor are electrically coupled to the terminal part,
the terminal part is coupled to a first end of the flexible printed circuit board, and
the flexible printed circuit board is bent, and the notch of the substrate is disposed at a location overlapping the light source.

2. The detection device according to claim 1, wherein the first optical sensor and the second optical sensor share the lower buffer layer, the active layer, the upper buffer layer, and the upper electrode.

3. The detection device according to claim 1, wherein the electronic components are mounted on a surface of the flexible printed circuit board opposite the light source.

4. The detection device according to claim 1, wherein the electronic components are mounted on a surface of the flexible printed circuit board on which the light source is mounted.

5. The detection device according to claim 1, wherein the substrate and the flexible printed circuit board are accommodated in a ring-shaped housing.

6. A detection device comprising:
a substrate provided with an optical sensor;
a terminal part provided at one end in a first direction of the substrate; and
a flexible printed circuit board on which a light source and a plurality of electronic components are mounted, wherein
a lower electrode, a lower buffer layer, an active layer, an upper buffer layer, an upper electrode, and a sealing film are stacked on the substrate in the order as listed, in the optical sensor,
each of the lower electrode and the upper electrode of the optical sensor is electrically coupled to the terminal part,
the terminal part is coupled to a first end of the flexible printed circuit board, and
the flexible printed circuit board is bent, and another end in the first direction of the substrate is located in a position adjacent to the light source.

7. The detection device according to claim 6, wherein the electronic components are mounted on a surface of the flexible printed circuit board opposite the light source.

8. The detection device according to claim 6, wherein the electronic components are mounted on a surface of the flexible printed circuit board on which the light source is mounted.

9. The detection device according to claim 6, wherein the substrate and the flexible printed circuit board are accommodated in a ring-shaped housing.
